(19) United States
(12) Patent Application Publication
(10) Pub. No.: US 2024/0261195 A1
(43) Pub. Date: Aug. 8, 2024

(54) COSMETIC CONCENTRATE IN THE FORM OF A FILM

(71) Applicant: BEIERSDORF AG, Hamburg (DE)

(72) Inventors: Eefje KOOP, Norderstedt (DE); Cecile RATSCHOW, Hamburg (DE)

(21) Appl. No.: 18/566,724

(22) PCT Filed: May 10, 2022
(86) PCT No.: PCT/EP2022/062618

§ 371 Date: Dec. 4, 2023

(30) Foreign Application Priority Data
Jun. 22, 2021 (DE) 10 2021 206 400.0

(51) Int. Cl. A61K 8/02; A61K 8/46; A61K 8/81; A61Q 19/10

(52) U.S. Cl. CPC A61K 8/0204; A61K 8/463; A61K 8/8129; A61Q 19/10

(57) ABSTRACT

The present invention relates to a cosmetic concentrate suspended in a film for producing a cleaning agent by dissolving in water. Preferably polyvinyl alcohol is selected as the binder.

Cosmetic products generally not only contribute to a beautiful and attractive appearance, but their effects make a crucial contribution to increased self-esteem and people's wellbeing. Accordingly, a wide variety of cosmetic products are used for daily cleansing and care of human skin.

Conventional cleansing formulations are usually sold in the form of a liquid, aqueous formulation. If the ingredients of such a formulation are analyzed, it can be established that the formulation comprises a significant amount of water. Typical examples of shower gels comprising 20% by weight to 95% by weight water are disclosed in WO1994010975A1, WO1993021900A1 and WO2002005758A2.

Accordingly, significant amounts of water are transported from the supplier to the stores and on to the customer to provide the customer with the cleansing formula of their choice. Considering the fact that most customers' homes are connected to the public water supply, it does not seem sensible from an ecological point of view to send water to a place that in principle has unlimited access to water. By dispensing with water in the formulations and shipping a concentrate in liquid or solid form, transport costs and transport-related emissions of greenhouse gases can thus be reduced.

Novel concepts include, for example, the dispatch of concentrates and mixing with water at the point of sale. Other approaches use solid cleansing compositions in the form of a bar. The latter formulations are applied by the customer like a bar of soap, for example by rubbing the bar over the body. However, the use of a solid bar for applying active cleansing ingredients to the human body is often not desirable for customers who are accustomed to applying cleansing formulations in liquid form.

Consequently, environmentally friendly cosmetic cleansing preparations may also be supplied to the consumer in the form of concentrates. The consumer dissolves the concentrates in water in predetermined proportions, so that a cosmetic cleansing preparation is produced. This form of application saves water in the production of concentrates. This leads to a lower environmental impact by minimizing weight, as the consumer can usually add water at home.

Known concentrates can be in liquid or solid form. However, in the case of a solid form, a distinction has to be made between pulverulent preparations, which are free-flowing, and preparations which are in the form of a single, solid piece. The solid piece can be obtained by compression of a pulverulent preparation or by melting and cooling the preparation to give a solidified cohesive structure.

A disadvantage of known products in which a pulverulent preparation is wholly encapsulated in a water-soluble film is that the product can be scattered if the film is inadvertently torn. A disadvantage of known products in which a liquid preparation is wholly encapsulated in a water-soluble film is that the product may leak if the film is inadvertently torn.

Consequently, known products have a considerable disadvantage in terms of handling.

Surprisingly, it has now been found that the disadvantages of the prior art could be eliminated or alleviated by the present invention.

The present invention relates to a cosmetic cleansing product in solid form comprising,
a) a binder, and
b) a solid cleansing concentrate containing one or more surfactants that are solid under standard conditions,
   wherein the cleansing concentrate contains less than 7% ingredients that are liquid under standard conditions, the indication referring to the total weight of the cleansing concentrate,
      characterized in that the cleansing concentrate is suspended in the binder.

The binder is advantageously water-soluble.

In this case, the cleansing product according to the invention is added to water, so that the dissolution of the binder results in the cleansing concentrate suspended therein being dissolved in the surrounding water.

In the context of the present invention “suspended in the binder” signifies that the cleansing product is present uniformly distributed throughout the entire binder.

Surprisingly, this makes it possible to provide products in sheet form, which can be easily measured out by cutting them to the required size. Consequently, the cleansing product according to the invention can be used significantly more flexibly than, for example, pre-packaged cleansing pods. Cleansing pods are characterized in that the binder encloses the entirety of the cleansing concentrate in the form of a single capsule. Consequently, the cleansing concentrate is not suspended in the binder. Consequently, a metered solution of the cleansing concentrate is not possible.

Surprisingly, it was possible to form a cohesive cosmetic cleansing product in combination with the binder when using the solid cleansing concentrate described.

If percentages by weight (% by weight) should be given below without reference to a particular composition or specific mixture, then these figures always refer to the total weight of the cleansing concentrate. If ratios of components/substances/substance groups should be disclosed below, these ratios refer to ratios by weight of the components/substances/substance groups specified.

If ranges of percentages by weight are given below for the constituents of the cleansing concentrate, the disclosure of the present application also includes all individual values in increments of 0.1% by weight within these weight percentage ranges.

In the context of the present disclosure, the formulations “according to the invention”, “advantageous according to the invention”, “advantageous in the context of the present invention” etc. always refer both to the cleansing concentrate according to the invention and to the use and the method according to the invention.

Unless specified otherwise, all experiments were carried out under standard conditions. The term “standard conditions” signifies 20° C., 1013 hPa and a relative humidity of 50%.

If the term skin is used, this refers preferably to human skin.

All substances cited below are named by their INCI declaration or by a unique chemical name.

Emulsifiers are understood to mean all substances which are listed in the International Cosmetic Ingredient Dictionary and Handbook, Thirteenth Edition 2010, (ISBN 1-882621-47-6) under the designation “emulsifying agent”. Surfactants are understood to mean all substances which are listed in the International Cosmetic Ingredient Dictionary and Handbook, Thirteenth Edition 2010, (ISBN 1-882621-47-6) under the designation “surfactant”.

Where viscosity values are given in this disclosure, all values refer to a measurement at 25° C. in a 150 ml wide-neck bottle (VWR No.: 807-001) using a Rheomat R 123 from proRheo. The Rheomat R 123 from proRheo GmbH is a rotational viscometer, i.e. a measurement body rotates in the substance to be measured. The force is measured that is required to rotate the measurement body in the sample at a predefined speed. The viscosity is calculated from this torque, the rotational speed of the measurement body and the geometric dimensions of the measuring system used. The measurement body used is the No. 1 measurement body (Article No. 200 0191), suitable for a viscosity range up to 10,000 [mPa·s], rotational speed range 62.5 min−1.

The binder is advantageously selected from the group of water glass, emulsion polymer isocyanate, formaldehyde resin, water-based acrylate and styrene acrylate dispersion, methylcellulose, methylcellulose ester, linseed oil, glutin paste, casein paste, starch paste, pozzolan, polyvinyl alcohol, casein, casein paste, lime casein, epoxy and polyester resin, isocyanate, polyurethane, PVAc paste, UF, polyvinyl alcohol, W paste, HF paste and phenol paste or combinations thereof. Preference is given to binders of biogenic origin. Particularly preferably selected from the group of methylcellulose, methylcellulose ester, linseed oil, glutin paste, casein paste, polyvinyl alcohol, starch paste, pozzolan, casein, casein paste, lime casein.

It is particularly preferred that the binder is selected from the group of PVAc paste, UF, methylcellulose ester, polyvinyl alcohol, starch paste, casein paste, W paste and HF paste or combinations thereof.

The binder selected is particularly preferably polyvinyl alcohol. Advantageously, no further binders are present in addition to polyvinyl alcohol.

Advantageously, the cosmetic product is in the form of a film, wherein the binder forms a continuous structure or film in which the cleansing concentrate is suspended. By adding water in excess to the product according to the invention, the film is dissolved so that the suspended cleansing concentrate is released.

Films composed of polyvinyl alcohol have a particularly advantageous water solubility. This means that the film disintegrates particularly rapidly.

It was surprising to those skilled in the art that the cleansing concentrate can be easily suspended in the binder to form a film. This was the case despite the possibility of small amounts of water in the concentrate.

According to the invention, the cosmetic cleansing concentrate comprises at least one surfactant that is solid under standard conditions. Advantageous surfactants are selected from the group of Sodium Myristoyl Glutamate, Sodium Lauroyl Glutamate, Sodium Stearoyl Glutamate, Sodium Cocoyl Glutamate, Sodium Coco Sulfate, Disodium Lauryl Sulfosuccinate and Sodium Methyl Cocoyl Taurate. It is particularly advantageous if Disodium Lauryl Sulfosuccinate and/or Sodium Coco Sulfate are present in the cleansing concentrate.

It is advantageous in the context of the present invention if the proportion of surfactants that are solid under standard conditions is from 0.1 to 50% by weight, preferably from 15 to 45% by weight and particularly preferably from 25 to 44% by weight, based on the total weight of the cleansing concentrate.

It is advantageous in the context of the present invention if the proportion of surfactants that are solid under standard conditions selected from the group of Sodium Myristoyl Glutamate, Sodium Lauroyl Glutamate, Sodium Stearoyl Glutamate, Sodium Cocoyl Glutamate, Sodium Coco Sulfate, Disodium Lauryl Sulfosuccinate and Sodium Methyl Cocoyl Taurate is from 0.1 to 50% by weight, preferably from 15 to 45% by weight and particularly preferably from 25 to 44% by weight, based on the total weight of the cleansing concentrate.

It is particularly advantageous in the context of the invention if the proportion of Disodium Lauryl Sulfosuccinate and/or Sodium Coco Sulfate is from 0.1 to 50% by weight, preferably from 15 to 45% by weight, preferably from 25 to 44% by weight and particularly preferably from 30 to 43% by weight, based on the total weight of the cleansing concentrate.

Disodium Lauryl Sulfosuccinate is advantageously present at a proportion of 8 to 30% by weight, preferably 10 to 25% by weight and particularly preferably from 12 to 20% by weight, based on the total weight of the cleansing concentrate.

Sodium Coco Sulfate is advantageously present at a proportion of 12 to 34% by weight, preferably 14 to 30% by weight and particularly preferably from 18 to 25% by weight, based on the total weight of the cleansing concentrate.

Furthermore, it is advantageous if Disodium Lauryl Sulfosuccinate and Sodium Coco Sulfate are present and further advantageously the ratio by weight of Disodium Lauryl Sulfosuccinate to Sodium Coco Sulfate is from 1.5:1 to 1:2, preferably 1:1 to 1:1.5 and particularly preferably 1:1.1 to 1:1.4.

Furthermore, it is advantageous according to the invention if Caesalpinia spinosa Gum and/or Cellulose Gum are present in the cleansing concentrate. Advantageously, the total proportion of Caesalpinia spinosa Gum and/or Cellulose Gum is from 0.1 to 30% by weight, preferably from 0.2 to 25% by weight, further preferably from 0.3 to 20% by weight and particularly preferably from 10 to 20% by weight, based on the total weight of the concentrate.

If Caesalpinia spinosa Gum is present, the proportion of Caesalpinia spinosa Gum is advantageously from 0.1 to 30% by weight, preferably from 0.2 to 25% by weight and particularly preferably from 0.3 to 20% by weight, based on the total weight of the concentrate.

If Cellulose Gum is present, the proportion of Cellulose Gum is advantageously from 0.1 to 30% by weight, preferably from 0.2 to 25% by weight and particularly preferably from 0.3 to 20% by weight, based on the total weight of the concentrate.

Accordingly, embodiments comprising Caesalpinia spinosa Gum are preferred, wherein further polymers are advantageously present at proportions of less than 10% by weight. It is particularly advantageous if only Caesalpinia spinosa Gum is present.

Furthermore, it is preferred if the concentrate is free from Xanthan Gum, Sphingomonas Ferment Extract, Dehydroxanthan Gum and/or Hydroxypropyl Guar.

Furthermore, it is preferred if the concentrate is free from polymers which are obtained from a homopolymerization or copolymerization with vinyl pyrrolidone, acrylic and/or methacrylic acid. Such polymers no longer meet today's ecological requirements, since the biodegradability thereof has not been conclusively clarified.

Free from in the context of the present invention signifies that the total proportion of said substances is less than 0.05% by weight and particularly preferably 0% by weight, the figures referring to the total weight of the concentrate.

Furthermore, it has been shown, surprisingly, that the addition of Maltodextrin significantly increases the creaminess of a cleansing product produced from the concentrate after foaming with a squeeze foamer, without causing precipitates in the cleansing product.

Accordingly, it is advantageous if the concentrate comprises Maltodextrin. If Maltodextrin is present, it is further advantageous if the proportion of Maltodextrin is from 0.1 to 20% by weight, preferably from 2 to 14% by weight and particularly preferably from 3 to 9% by weight, based on the total weight of the concentrate.

Furthermore, it is advantageous if the concentrate comprises Magnesium Carbonate Hydroxide. If Carbonate Hydroxide is present, it is further advantageous if the proportion of Carbonate Hydroxide is from 0.5 to 15% by weight, preferably from 1 to 8% by weight and particularly preferably from 2 to 6% by weight, based on the total weight of the concentrate.

Furthermore, it is advantageous if the concentrate comprises Sodium Benzoate. If Sodium Benzoate is present, it is further advantageous if the proportion of Sodium Benzoate is from 0.1 to 30% by weight, preferably from 5 to 25% by weight and particularly preferably from 10 to 20% by weight, based on the total weight of the concentrate.

Furthermore, it is advantageous if the concentrate comprises Hydroxyacetophenone. If Hydroxyacetophenone is present, it is further advantageous if the proportion of Hydroxyacetophenone is from 0.1 to 12% by weight, preferably from 1 to 10% by weight and particularly preferably from 3 to 7% by weight, based on the total weight of the concentrate.

Furthermore, it is advantageous if the concentrate comprises Citric Acid. If Citric Acid is present, it is further advantageous if the proportion of Citric Acid is from 0.1 to 30% by weight, preferably from 5 to 25% by weight and particularly preferably from 10 to 23% by weight, based on the total weight of the concentrate.

Furthermore, it is advantageous if the concentrate comprises Sodium Citrate. If Sodium Citrate is present, it is further advantageous if the proportion of Sodium Citrate is from 0.1 to 10% by weight, preferably from 1 to 5% by weight and particularly preferably from 2 to 4% by weight, based on the total weight of the concentrate.

Furthermore, it is advantageous if the concentrate comprises DMDM Hydantoin. Chlorphenesin, Iodopropyl Butylcarbamate and/or Hexamidine Diisethionate.

If DMDM Hydantoin is present, it is advantageous if the proportion of DMDM Hydantoin is from 0.5 to 12% by weight, based on the total weight of the concentrate.

If Chlorphenesin is present, it is advantageous if the proportion of Chlorphenesin is from 0.5 to 13% by weight, based on the total weight of the concentrate.

If Iodopropyl Butylcarbamate is present, it is advantageous if the proportion of Iodopropyl Butylcarbamate is from 0.1 to 13% by weight, based on the total weight of the concentrate.

If Hexamidine Diisethionate is present, it is advantageous if the proportion of Hexamidine Diisethionate is from 0.1 to 5% by weight, based on the total weight of the concentrate.

It is advantageous if the proportion of additives that are liquid under standard conditions is less than 15% by weight, preferably less than 10% by weight and especially less than 7% by weight, based on the total weight of the concentrate.

If water is present in the concentrate as liquid additive, the proportion of water is preferably less than 5% by weight and particularly preferably less than 2% by weight, based on the total weight of the concentrate. In particular, this water may be introduced via ingredients that are not completely anhydrous. Accordingly, it is particularly advantageous if no free water is added to the concentrate.

Free water is water that is in pure form, i.e. without being added as a constituent of other ingredients.

Furthermore, it is advantageous if the concentrate comprises one or more perfumes. In the case of liquid perfumes, it is preferable if the proportion of liquid perfumes does not exceed 7% by weight, based on the total weight of the concentrate.

Furthermore, the concentrate may advantageously comprise further ingredients, such as advantageously colorants or active ingredients. Advantageously, no water-insoluble ingredients under standard conditions are present.

Furthermore, the concentrate is preferably free from soaps. It is preferable if no further surfactants are present.

In one embodiment of the invention, the ratio by weight of binder to cleansing concentrate is in the range from 99:1 to 1:99, advantageously from 90:10 to 10:90, advantageously from 80:20 to 20:80, advantageously from 70:30 to 30:70, advantageously from 60:40 to 40:60.

In one embodiment of the invention, the ratio by weight of binder to cleansing concentrate is in the range from 99:1 to 1:1, advantageously from 50:1 to 2:1, advantageously from 30:1 to 3:1, advantageously from 20:1 to 4:1, advantageously from 15:1 to 5:1.

In one embodiment of the invention, the ratio by weight of cleansing concentrate to binder is in the range from 99:1 to 1:1, advantageously from 50:1 to 2:1, advantageously from 30:1 to 5:1, advantageously from 20:1 to 10:1, advantageously from 18:1 to 12:1.

### COMPARATIVE EXPERIMENTS AND EXAMPLES

The examples below are intended to elucidate the present invention without limiting it. Unless otherwise stated, all amounts, proportions, and percentages are based on the weight and total amount and on the total weight of the preparations.

The following tables show different concentrates (Ex. 1 to Ex. 11).

| Ingredients | Ex. 1 | Ex. 2 | Ex. 3 | Ex. 4 | Ex. 5 |
|---|---|---|---|---|---|
| Citric Acid | 15.63 | 15.63 | 15.63 | 15.63 | 15.63 |
| Sodium Benzoate | 15.63 | 15.63 | 15.63 | 15.63 | 15.63 |
| 99% Sodium Citrate + | 2.34 | 2.34 | 2.34 | 2.34 | 2.34 |
| 1% Aqua | | | | | |
| Magnesium Carbonate | 2.34 | 2.34 | 2.34 | 2.34 | 2.34 |
| Hydroxide | | | | | |
| 95% Disodium Lauryl | 15.63 | 15.63 | 15.63 | 15.63 | 15.63 |
| Sulfosuccinate + 5% | | | | | |
| Aqua | | | | | |
| Maltodextrin | 7.81 | 7.81 | 7.81 | 7.81 | 7.81 |
| Hydroxyacetophenone | 5.47 | 5.47 | 5.47 | 5.47 | 5.47 |
| 97% Sodium Coco | 19.52 | 19.52 | 19.52 | 19.52 | 19.52 |
| Sulfate + 3% Aqua | | | | | |
| Caesalpinia Spinosa | 15.63 | | | | |
| Gum | | | | | |
| Cellulose Gum | | 15.63 | | | |
| Dehydroxanthan Gum | | | 15.63 | | |
| Hydroxypropyl Guar | | | | 15.63 | |
| Xanthan Gum | | | | | 15.63 |

| Ingredients | Ex. 6 | Ex. 7 | Ex. 8 | Ex. 9 | Ex. 10 | Ex. 11 |
|---|---|---|---|---|---|---|
| Citric Acid | 23.87 | 25.64 | 16.65 | 20.80 | 19.67 | 21.65 |
| Sodium Benzoate | 20.06 | 15.54 | 18.34 | 17.33 | 17.95 | 15.64 |
| 99% Sodium Citrate + 1% Aqua | 3.80 | 1.30 | 4.86 | 2.60 | 3.97 | 3.64 |
| Magnesium Carbonate Hydroxide | 3.55 | 8.30 | 6.63 | 5.20 | 4.86 | 4.80 |
| 95% Disodium Lauryl Sulfosuccinate + | 16.05 | 13.64 | 17.88 | 17.33 | 10.67 | 20.67 |
| 5% Aqua | | | | | | |
| Maltodextrin | 3.85 | 1.66 | 5.34 | 3.47 | 6.84 | 4.09 |
| Hydroxyacetophenone | 5.93 | 2.82 | 4.37 | 6.07 | 6.90 | 7.49 |
| 97% Sodium Coco Sulfate + 3% Aqua | 15.88 | 22.34 | 20.46 | 21.65 | 23.59 | 17.37 |
| Caesalpinia Spinosa Gum | 1.00 | 0.76 | 0.15 | 0.35 | 0.65 | 0.70 |
| Fragrance | 5.90 | 6.20 | 4.55 | 5.20 | 4.90 | 3.95 |
| Aqua | to 100 | to 100 | to 100 | to 100 | to 100 | to 100 |

In addition, a mixture of 100 parts by weight PVA (polyvinyl alcohol), 5 parts by weight glycerol as softener and 15 parts by weight of one of the above concentrates of the examples (Ex. 1 to Ex. 11) was added to water such that a 5% aqueous solution was obtained. The resulting solution was poured onto a polyester film and dried at room temperature. The resulting film was then detached from the polyester film. The film thus obtained was subjected to heat treatment at 100° C. for 10 minutes.

Thus, a product can be obtained which comprises a pulverulent concentrate suspended in PVA.

The film obtained can be added to water by the consumer, whereby the film dissolves and releases the concentrate. A cosmetic cleansing product is thus obtained. Dosing is carried out by selecting the size of the film.

1.-11. (canceled)

12. A cosmetic cleansing product, wherein the product is present in solid form and comprises
(a) a binder, and
(b) a solid cleansing concentrate comprising one or more surfactants that are solid under standard conditions,
the cleansing concentrate being suspended in the binder and comprising less than 15% by weight, based on a total weight of the concentrate, of ingredients that are liquid under standard conditions.

13. The cleansing product of claim 12, wherein the binder is water-soluble.

14. The cleansing product of claim 12, wherein the binder is selected from one or more of PVAc paste, UF, methylcellulose ester, polyvinyl alcohol, starch paste, casein paste, W paste, HF paste.

15. The cleansing product of claim 12, wherein the binder comprises polyvinyl alcohol.

16. The cleansing product of claim 15, wherein no further binders are present.

17. The cleansing product of claim 12, wherein the product is present in the form of a film in which the binder forms a continuous structure having the cleansing concentrate suspended therein.

18. The cleansing product of claim 12, wherein concentrate comprises at least one surfactant selected from Sodium Myristoyl Glutamate, Sodium Lauroyl Glutamate, Sodium Stearoyl Glutamate, Sodium Cocoyl Glutamate, Sodium Coco Sulfate, Disodium Lauryl Sulfosuccinate, Sodium Methyl Cocoyl Taurate.

19. The cleansing product of claim 12, wherein the concentrate comprises Disodium Lauryl Sulfosuccinate and/or Sodium Coco Sulfate.

20. The cleansing product of claim 12, wherein a concentration of surfactants that are solid under standard conditions is from 0.1% to 50% by weight.

21. The cleansing product of claim 20, wherein the concentration of surfactants that are solid under standard conditions is from 15% to 45% by weight.

22. The cleansing product of claim 20, wherein the concentration of surfactants that are solid under standard conditions is from 25% to 44% by weight.

23. The cleansing product of claim 12, wherein a concentration of additives that are liquid under standard conditions is less than 15% by weight, based on a total weight of the concentrate.

24. The cleansing product of claim 23, wherein the concentration of additives that are liquid under standard conditions is less than 10% by weight.

25. The cleansing product of claim 23, wherein the concentration of additives that are liquid under standard conditions is less than 7% by weight.

26. The cleansing product of claim 12, wherein a concentration of water as liquid additive is less than 5% by weight, based on a total weight of the concentrate.

27. The cleansing product of claim 26, wherein a concentration of water as liquid additive is less than 2% by weight.

28. The cleansing product of claim 12, wherein a weight ratio of binder to cleansing concentrate is from 99:1 to 1:1.

29. The cleansing product of claim 28, wherein the weight ratio is from 50:1 to 2:1.

30. The cleansing product of claim 28, wherein the weight ratio is from 30:1 to 3:1.

31. The cleansing product of claim 28, wherein the weight ratio is from 20:1 to 4:1.
